(19) United States
(12) Patent Application Publication
(10) Pub. No.: US 2011/0112554 A1
(43) Pub. Date: May 12, 2011

(54) SUTURING DEVICE

(76) Inventor: Arkady Veniaminovich Dubbrovsky, Moscow (RU)

(21) Appl. No.: 12/997,629

(22) PCT Filed: Jun. 11, 2009
(86) PCT No.: PCT/RU09/00299

§ 371 Date: Dec. 13, 2010

(30) Foreign Application Priority Data
Jun. 18, 2008 (RU) 2008123944

(51) Int. Cl. A61B 17/04

(52) U.S. Cl. 606/144

(57) ABSTRACT

The invention relates to medical devices. Suturing device comprises a body, two working jaws, suturing elements and a needles receiver after suturing. One of the jaws is stationary and the second relates to the body with the ability to move relative to the fixed jaw parallel to its work surface. As suturing elements direct atraumatic needles are used. The after suturing needles receiver is equipped with a needles fixation device and a device for tensioning and securing threads. Atraumatic needles are installed in a fixed jaw perpendicular to its work surface. Fixed jaw is provided with the pusher moving needle in a rectilinear direction. After suturing needles receiver is installed on the mobile jaw. The invention minimizes the trauma of biological tissues during the suturing and increases convenience when using the device.

### TECHNICAL FIELD

The invention relates to medical equipment and more precisely concerns a suturing device, designed primarily for suturing biological tissues, but can be used in other areas.

### BACKGROUND ART

Surgical suturing devices are known (Russian apparatus UKB, UKL, UTL, UDO, and also the suturing devices of the company USSC and of the company Ethicon). These devices are using for stitching tissue metallic clips, which are not absorbed and are stored in the tissues, as a foreign, even though the inert material. Created absorbable staples are used almost exclusively in gynecology because of its size (the device PolyCS of the company USSC).

Designs of sewing devices using atraumatic needles with absorbable threads are known, but all known devices have not found propagation in practice because of the lacks inherent in their design.

In particular, in the apparatus Patent RU 2074651 C1 and RU 2076639 C1 of 1993, are used atraumatic needles, which, given the design features of these devices, are deformed in the process of suturing. As a result, at the time of pulling the needle out of stitched tissues, there is a residual elastic deformation of the blunt end of the needle, which is traumatic for the sewn tissues.

In the patent RU 2310405 C2 of 2004 the option is offered of sewing device containing a body, two working jaws one of which is stationary, while the second is connected to the body and can move relative to the fixed jaws parallel to the work surface, sewing elements—direct atraumatic needles, and after sewing needles receiver, equipped with a device for fixing needle and thread tension and consolidate after suturing. However, this apparatus has a complex design and is unsuitable for use in surgical practice.

The most user-friendly design have staplers, described in patent SU, A, 235911, SU, A, 266138, SU, A, 674747, SU, A, 886,900. In particular, in the patent SU, A, 886,900 is disclosed suturing device, containing a body, two working jaws at its distal end, perpendicular to the body, one of which is stationary, while the second is connected to the body to move relative to the fixed jaw parallel to the work surface, sewing elements and controls means of the relative position of working jaws and pulling a suturing element through tissue. However, this suturing device, as well as other above-mentioned devices, having a similar design, are adapted for use as a sewing elements metal clips, the shortcomings of which have already been discussed above.

### SUMMARY OF INVENTION

A basis of invention is to develop a suturing device having a construction which would be adapted for use as a suturing element with direct atraumatic needles and thus would be fairly simple and easy to use.

The problem is solved so that the suturing device, containing a body, two working jaws, one stationary, while the second relates to the body to move relative to the fixed jaw parallel to the work surface, suturing elements, which are mainly the direct atraumatic needles, the needles receiver after sewing, equipped with a device for fixation needles and tension and fixing filaments tool, according to the invention, direct atraumatic needles are installed in a fixed jaw perpendicular to the work surface, where the fixed jaw is provided with a pusher moving needle in a straight direction, and the needle receiver after the suturing is installed on the mobile jaw.

In a preferred variant of the realization the atraumatic needles are fixed in pairs (a needle in each end of the thread or one thread with branches 3-4 mm or more with a needle at each end) in two rows on the movable frame in a removable cassette is inserted in through the slot, made in fixed jaw and needle pusher is interacting by its working surface with needle blunt ends and with the moving frame.

It is possible that the receiver of needles will be made in the form of a hollow shell, in the wall facing the fixed jaw, will be carried out slots, adapted for the transmission of the needles, and placed inside the receiver after sewing needles fixation device, related by a rod with a handle of fixation device, located at the proximal end of the shell.

In one of the options to implement a device for needles fixation after sewing contains pad of elastic material placed over the bridge between the slots and clamping element attached to the end of the rod of the fixating device, in contact with an elastic pad to ensure its compression and expansion in the transverse direction when the forward movement of the said rod, while the stopper is provided for fixing the clamping element in the position of the elastic pad compression.

In another embodiment of the needle fixation there are three plates mounted in a hollow body of the needle receiver unit on its side with the slotted holes perpendicular to it, the middle plate located on the dam between the slots, and the exterior plates are on the opposite sides of the holes area. All three plates at the ends opposed to the ones contacted with the receiver body wall, are connected to each other with an axis, which is fixed on the end walls of the needles receiver.

Fixation of the outer plates on two parallel axes is possible. In this case, the inner plate is T-shaped, and the axes of the outer plates are located on the edges of the transverse strips of the interior plate. The ends of the axis or of the two parallel axes are mounted on the end walls of the receiver unit. The thickness of the middle plate is equal to the distance between the rows of needles. This plate is located in the center of the receiver body, parallel to the rows of needles, so that it is between the rows of needles after sewing. The end plates are freely swinging on the longitudinal axes (or longitudinal axis). Between them there is a spring, which is extending them. The end plates have extensions on their ends, covered with an elastic material. Two rows of stitched needles fall in the needle receiver into the space between the middle and the end plates. To the top of the plates is moving an U-shaped clamping element, approaching the end plates to the middle one, thus there is a strong fixation of the ends of stitched needles, located in the intervals between the plates. For a more uniform compression of the set of needles, which have not always exactly the same diameter, the inner surfaces of the outer plates may be covered with a dense rubber, synthetic elastic material or covered with “soft” metals (copper, aluminum, etc.). With sufficient compression of the plates, the U-shaped element is fixed by a stopper in this position, after which the rod of the fixing means goes up, removing the needle receiver with the stitched needles fixed between the plates.

Other variants of the needles fixation means are possible. For example, on the bottom of the hollow body of the needles receiver three plates can be placed parallel to the bottom of the receiver. There are holes for the needles in the plates, these hole have to coincede with the holes in the bottom of the moving jaw and the holes in the bottom of the receiver. The middle plate is mobile, it can be made of a soft metal (copper, aluminum, etc.) and rests on the cam at the end of the rod of the fixation mean, controlling the rise of the fixative plates and the needles receiver body. After tissue suturing, the ends of the needles fall through the bottom of the needles receiver into these holes in the plates. By turning the rod of the fixing device around its longitudinal axis the eccentric is rotated. Eccentric shifts the middle plate, while the ends of stitched needles are firmly fixed. Since then the stock of the fixation device ascend, lifting the needle receiver body together with fixed needles.

The variant of the implementation of needles fixing device from the two plates when the bottom of the needles receiver body is quite solid and can replace the bottom plate, without inducing the movement of blunt side of the needle when it is fixed by moving plate.

The variant of the implementation of the stitched needle fixing device with a wide wedge compressing the stitched needle to the walls of the body of needles receiver is possible.

The variant strain of sharp ends of needles, which goes after flashing in special hole, like the known staplers. In order to reduce the force applied to the needles pusher for bending the sharp ends of needle and provide a secure fixation of needles for their extraction from tissues, persistent mobility of a plate with holes may be provided. In this case, rather small deviations of needles sharp ends, which goes into the hole, is enough. Then the rod of needles fixation device with the stopper plate moves down, increasing the angle of deviation of sharp ends of needle and pressing them to the appropriate section of the bottom of the needles receiver. In this case, should be provided a mechanism to avoid deviation of blunt ends of needles from a strictly rectilinear direction at the time of deformation of sharp ends of needles.

All of these embodiments of a device of fixing having sewed needles require strict compliance with the rectilinear motion of straight needles as if piercing the tissue, and when pulling the needle with the threads of sewed tissue, which is provided by construction of suturing device, according to the invention. Such movement of needles when sewing and pulling the needle from the tissue reduces tissue trauma to a minimum. In addition, the rectilinear motion of the threads in the sewed tissues completely eliminates sawing effect which can occur when pulling the thin strong threads with change of direction of its motion.

After fixing the ends of having sewed needles and complete removal of the needles from tissue, needle's ascending continues until a sufficient tension of the threads.

Then, a device of threads tension and fixation provides a fixation of stretched threads in pairs or in a different order. There are several options for this operation.

In one of the variants device for tightening and fixation of the threads after sewing can be done in the form of located on the surface of the clamping movable jaw layer film available for piercing with needles, between its layers are placed tightening loops, distributed, taking into account the pair wise coverage of all threads passing through the film when sewing, the ends of each loop are directed in opposite sites and fixed on the ring, passing around the perimeter of the mobile jaw and connected by the thrust with the lift ring.

In yet another variant on the lower surface of the movable clamping jaws have a pre-arranged loops of absorbable threads, better made of braided or twisted threads, the ends of which are collected in two thin tubes, that are located on both sides of the stitched threads. The variant, when all the ends of these loops on each side are attached in advance to one side of thread passed through the corresponding pipe, is possible. The tension of the two lateral threads leads to a simultaneous lengthening of the loop on the stretched threads.

It is also possible that the device for tension and consolidate of the threads after suturing was made as a plate mounted on the clamping surface of the movable jaw, and tubes of material, permeable for piercing needles when suturing, filled with biocompatible glue, located on lines of passing of the two rows of needles in suturing.

Other variants of the fixation of having sewed and stretched threads: twisting them into a thin rod (fishing line) and retraction of the rod with the folds of threads in a pipe, compression of the filaments between two ribbed surfaces, suturing through the multilayer stretched braided material, which after release is reduced in different directions, fusing them, bonding them together, or between the two glued surfaces, etc.

It is also possible to use threads from biodegradable polymer which are knotting themselves.

### BRIEF DESCRIPTION OF DRAWINGS

In the following the invention is explained by description of the concrete variants for its realization and by attached drawings in which:

FIG. 1 shows the suturing device, according to the invention, in general view from the side;

FIG. 2—the same as in FIG. 1, front view;

FIG. 3—the same as in FIG. 1, in the section;

FIG. 4—needle pusher, side view;

FIG. 5—upper movable jaw in the section, side view;

FIG. 6—cassette with needles and threads in the longitudinal section, side view;

FIG. 7—cassette with needles, cross-section;

FIG. 8—the tape in a fixed jaw, top view;

FIG. 9—a device of needles fixation, variant with an elastic strip, the cross-section;

FIG. 10—the same as in FIG. 9, a variant with three plates perpendicular to the bottom of the receiver with the needle slot openings, the cross-section;

FIG. 11—the same as in FIGS. 9 and 10, a variant with three plates arranged parallel to the bottom of the receiver with the needle slot openings, the cross-section;

FIG. 11a—the same as in FIG. 11, longitudinal section;

FIG. 12—the same as in FIGS. 9, 10 and 11, a variant with clenched needles, cross-section;

FIG. 13—the needles receiver body, side view;

FIG. 14—the same as in FIG. 12, in cross section;

FIG. 15—bottom view on the needles receiver body;

FIG. 16—a film with tightening loops and the ring for loops;

FIG. 17—a plate with tightening loops, the ends of which are collected in the tube;

FIG. 18—a plate with tubes of glue.

### BEST MODE FOR CARRYING OUT THE INVENTION

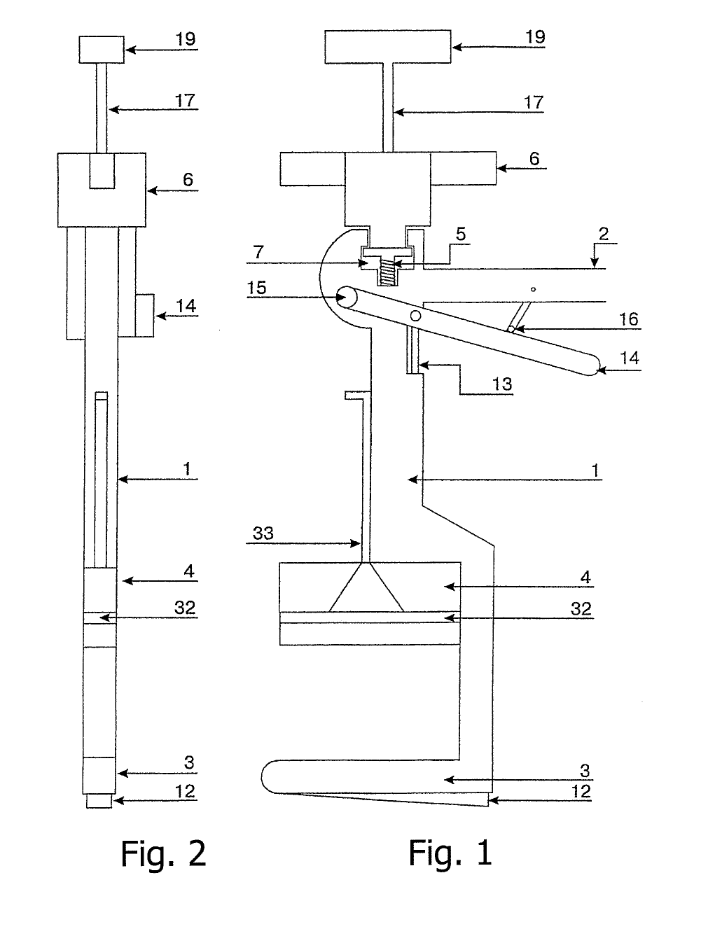

In FIGS. 1, 2 and 3 is presented in general form the side and front view of the suturing device, according to the invention.

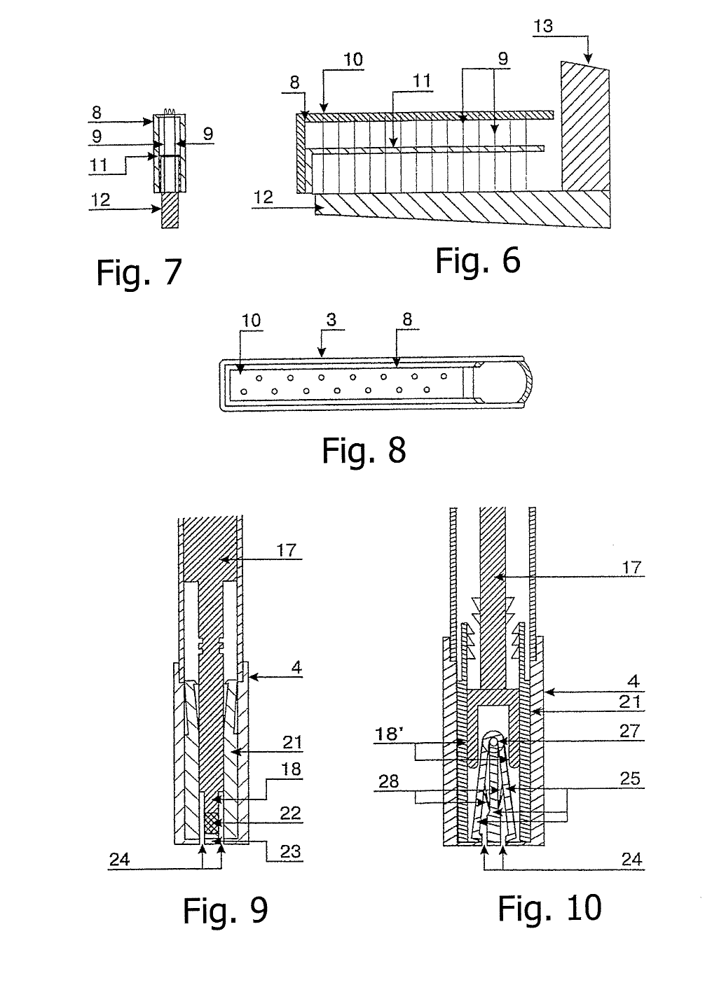

Suturing device comprises a body 1 with a stationary handle 2. The continuation of the body 1 in the distal part is a hollow fixed working jaw 3, oriented perpendicular to the body. Mobile working jaw 4 is connected with the body 1 with the possibility of reciprocating movement, relative to the body, parallel to the fixed clamping jaw 3. Mobile 4 jaw attached to the end of the rod 5 of the jaw, on the other end of which is made screw thread. Stem 5 of the jaw is installed in the longitudinal channel of the body 1 and is connected via a screw connection to the handle 6 of the control of movable jaw 4 position. At the lower end of the handle 6 there is a circular protrusion, located in the space of provided deepening 7 in the proximal part of the body 1, which limits the forward movement of the handle 6, but does not prevent its rotation. When rotating the handle 6, the stock 5 of the jaw and the movable jaw 4 move. In the fixed lower jaw 3 are made through the slot, in which is installed removable cassette 8 (FIGS. 6, 7 and 8) with needles 9. The upper clamping surface 10 of the cassette 8 can be covered with a thin absorbable film, which will provide additional hemostasis and increase the tightness of the suture. Inside the cassette 8 has a movable frame 11, in which there are two parallel rows of suturing elements are installed—direct atraumatic needles 9, oriented perpendicular to the working surface of the jaw 3 (clamping surface 10 of the cassettes 8). Under the magazine 8 is the pusher 12 which moves the needles 9 in a straight direction, it is communicating with its work surface with the blunt ends of the needles 9, for which in the working surface of the pusher 12 may be provided the deepening for needles 9 blunt ends with molded in it threads.

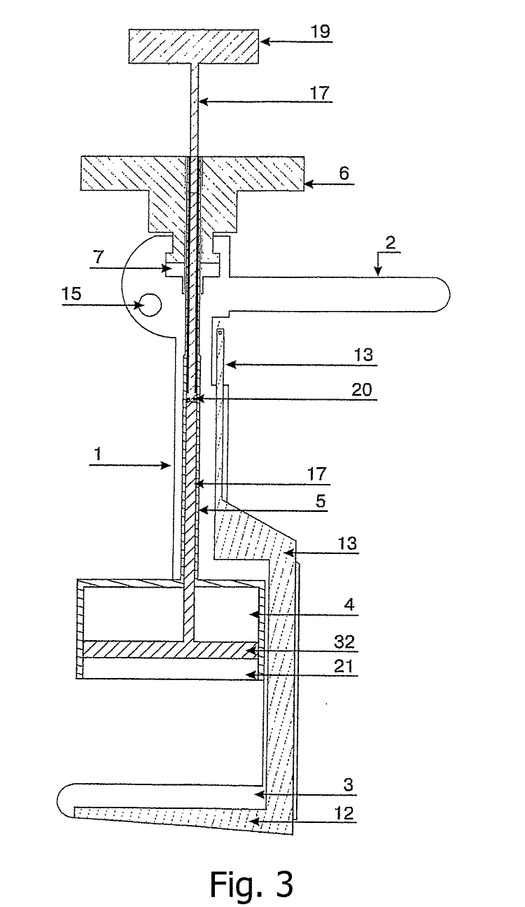

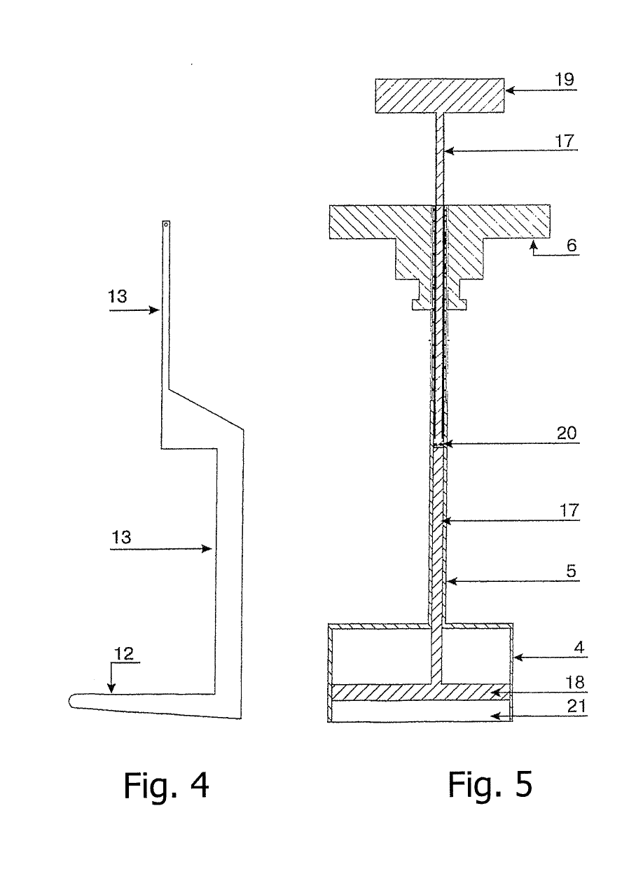

The pusher 12 for needles moving is fixed to the stock 13 (look at FIG. 4), which is installed with the possibility of translational movement in the body 1 cavity (FIG. 3). The upper end of the rod 13 is pivotally connected with a movable handle 14 (FIG. 1), which in turn, is connected through a hinge 15 with the body 1, which allows you to rotate it relative to the fixed handle 2 to control the position of suturing elements. To prevent pressing in wrong time the handle 14, there is a turning safeguard 16 mounted on the stationary handle 2.

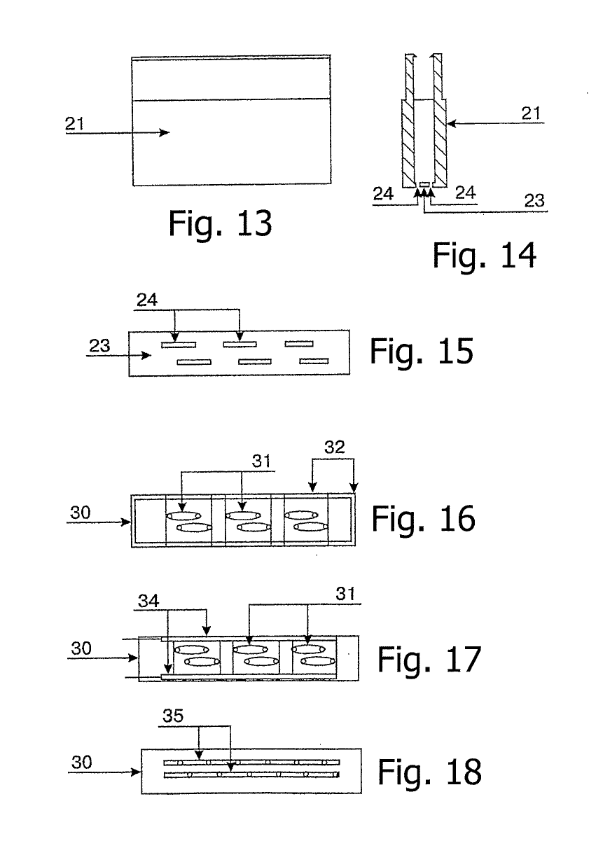

Stock 5 (FIGS. 3 and 5) of the mobile jaw 4 is hollow and inside of it is located the stock 17 of the needles fixation device, which at its distal end has the presser element 18, and at the proximal end—a control handle 19 to ménage the needles fixation device. Fixation device stock 17 consists of two parts connected with each other by a swivel 20. The upper part of stock 17 is provided with a screw thread, which corresponds to an internal screw thread, made in the wall of the canal in stock 5 of the jaw. This screw thread is opposite to the progress movement of a screw thread, located on the outer surface of the stock 5 of the jaw. Thanks to a screw thread at the top of the stock 17 latter can rotate and move within the stock 5 of the jaw. The lower part of stock 17, carrying the presser element 18 of the needle fixating device does not rotate, but is able to move steadily in the cavity of the stock 5 of the jaw, with the help of hinge joint 20. Inside the mobile jaw 4 is the needle's after suture receiver 21, equipped with a device of fixing the needle after suturing (see FIGS. 13 and 14). The needles receiver 21 has a hollow (preferably a box-like) shape, and needle's ends after suture get into it through the slots in the bottom of the receiver 21. There is a device of needles fixation in receiver 21. In FIGS. 9, 10, 11, 11a and 12 are four possible options for implementing the device of fixing the needles. In FIG. 9 is shown a version of a device of needles fixation containing a strip 22 of elastic material—a dense rubber or synthetic material of densely-elastic consistency, which is located above the bridge 23 between slots 24, made in the wall of the needles receiver 21 facing the fixed jaw, adapted to transmission of the appropriate needles. Over the elastic strip 22 is clamping element 18 in contact with the elastic strip 22. Needles after suture fall to the receiver through the slots 24 in the spaces between the side walls of the receiver 21 and the elastic strip 22. With forward movement of the rod 17 fixing device the clamping member 18 compresses the strip 22, causing it to expand in the horizontal direction, so that the needle is firmly pressed against the walls of the receiver 21. The upper part of the side walls of the receiver 21 body is refined so that it can bounce and has at its edges jagged. On the sides of the rod 17 of the fixation device there are also corresponding notches adapted to interact with the notches on the walls of the needles receiver 21 body. When the mentioned notches are engaged, a reverse movement of the rod 17 stops and the presser member 18 is fixed to the compression of the elastic strip 22. At the reverse movement up of the rod 17 of the fixing device, it raises the whole body of the receiver 21 along with fixed needles in it. FIG. 10 illustrates another embodiment of the needles fixation device. In the box-shaped body of needles receiver 21 there are three plates 25 perpendicular to the wall of the body with the slots 24 so that the middle plate 25 is located on the bridge 23, while the two edge plates—on opposite sides of the zone of the hole 24. All three plates 25, at the ends opposite to the contacting with the wall of the body of needles receiver 21, are interconnected by an axe 27, fixed to the end walls of the needles receiver 21. The edge plates 25 at its ends have extensions, covered with an elastic material, and spring-loaded relative to the middle plate with an elastic element—a spring 28. Stock 17 of the fixation device in this embodiment has at the end a U-shaped clamping member 18′, which, with the forward motion of the rod 17 may hang down on the three mentioned plates 25 and press the edge plates to the middle one. Having sewed needles enter the needles receiver 21 in between the middle plate 25 and the edge ones. When stock 17 is moved down, the presser member 18′ compresses all three plates 25, locking firmly the needles trapped between the plates. This embodiment also provides a lock to prevent slipping of plates 25 of the clamp U-shaped element 18′ during the reverse movement of the rod 17 of fixing device down. For this purpose, the upper edge of the needles receiver body 21 is provided with notches, snapping on the corresponding notch on the stock 17 when it moves downward. When the stock 17 is moved up, the whole body of the needles receiver 21 with fixed needles is rising.

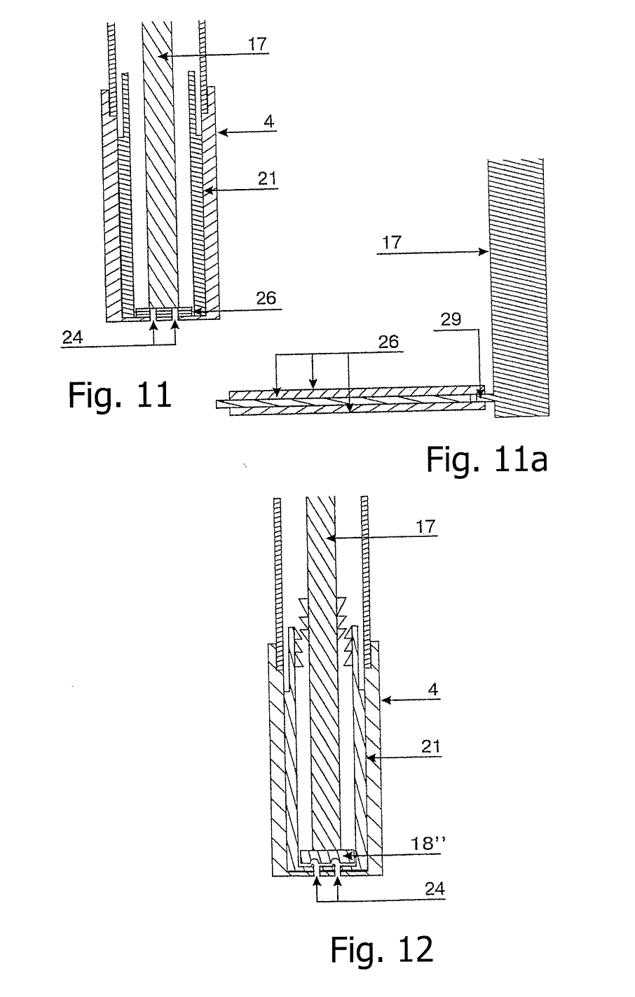

FIGS. 11 and 11a present yet another embodiment of the device fixing the ends of having sewed needles. At the bottom of the hollow body of needles receiver 21 with slotted openings 24 there are three plates 26 parallel to the bottom of the body of needles receiver 21. In plates 26 there are holes for needles, coinciding with the holes in the clamping surface of movable jaws. Middle plate 26 is movable; it can be made of a “soft” metal (copper, aluminum, etc.) and runs into the eccentric 29 at the end of the stock 17 of fixing device, controlling the rise of the fixing plates and the receiver body.

After suturing of the tissue the ends of needles fall through the bottom of the needles receiver 21 into these holes in the plates. When you turn the stock 17 of fixing device about its longitudinal axis, the mentioned above eccentric 29 rotates. Eccentric 29 moves the middle plate 26 (FIG. 11a), thus the ends of the having sewed needles are firmly fixed. After that stock 17 rises up, lifting the needles receiver body 21 with fixed needles.

At FIG. 12 is presented another embodiment of the device fixing the ends of having sewed needles. This option is somewhat similar to a mechanism known as staplers. At the bottom of a hollow needle receiver body 21 with slotted holes 24 is a plate 18″ with holes in which the ends of the having sewed needles 9 abut. With further pressure on the needle pusher 12, ends of the needles 9 are deformed so that the rise of the needles receiver 21 will remove needles from sutured tissues. However, in this version the needles pusher 12 should be significantly strengthened by, which will increase the size of the fixed jaws 3. In addition, while suturing the dense tissue, not enough deformed ends of the needles will not be able to prevent the needle slipping out of the needles receiver 21 in the rise. So, it is better to divided into two stages the process of the strain of all having sewed needles. At the beginning the sharp ends of having sewed needles 9 run into the holes and are slightly deformed. Then the stock 17 of the fixation device lowers the plate 18″ with holes, pressing it to the bottom of the receiver 21 of the needles. In this case, a slightly deformed sharp ends of the needles 9 are deformed further and pressed to the bottom of the receiver 21 of the needles. In this position, slipping of needles is impossible, and also there is no need to strengthen the pusher 12 of the needles. In this embodiment, stock 17 of fixing device is stopped in the bottom position as well as in the embodiment shown in FIG. 10.

FIG. 13 presents the needles receiver body 21, side view, and FIG. 14—cross-section of the receiver body 21.

FIG. 15 shows the bottom of the needle receiver 21 with slots 24 for needles 9 and the bridge 23 between them.

At FIGS. 16, 17 and 18 are embodiments of the device for tensioning and securing threads after sewing.

Device for tension and fixing threads after suturing, shown in FIG. 16, is done as a two-layer film 29′ which is available for needles piercing when suturing, and which is placed on clamping surface of movable jaws. Between the layers of the film 29′ are placed tightening loops 30, distributed taking into account the pair wise coverage of all threads passing through the film at the suturing. The ends of each loop 30 are directed in opposite directions and attached to the ring 31, passing around the perimeter of the mobile jaw and connected by rod 32 (FIG. 1) with a lift of ring 33. After suturing of tissue needles 9 initially pierce this plate with elements locking threads, and then enter the mobile jaw. By pulling the ends of each loop 30, two sewed threads, caught in the loop are tightened securely. When lifting ring 31 with the help of the lift 33 all attached to the ring 31 threads simultaneously are stretched and all tightening loops 30 are fixing firmly located within them sewed threads.

The second option of the device for tension and fixation of the threads after suturing is shown in FIG. 17. In this embodiment, the ends of the spanning loops 30 are attached to the side threads, located in the side tubes 34 on the two sides of the film 29′. The stretching of these lateral threads in tubes 34 leads to a simultaneous tightening of all spanning loops 30. In this version there is no need in a ring for fixing the threads and its lift. It is sufficient to conduct the lateral threads in the body 1 and attach them to a pull, the tension of which would lead to tightening of all the loops 30. It is possible to provide strong fixation of only one tube 34. In this case it is enough to pull one thread to tighten all the loops 30, but in this case, to prevent displacement of the loop in the direction of pulling thread, an emphasis of pulling thread is needed near the site of spanning loop.

The third option of the device for tension and fixation of threads after suturing is shown in FIG. 18. In this embodiment, between the layers of the film 29′ are two parallel tubes 35, filled with biocompatible glue. Tubes 35 are also made of material available for piercing by needles, and they are placed along the lines of the location of having sewed needle. Needles are piercing these tubes and then through them stretch sewed thread. Threads are glued with glue which is inside the tubes 35.

The whole sewing process goes as follows. Under the sewed tissues is fed immobile jaw 3. When turning the screw knob 6, the movable jaw 4 is dipped and brought to a fixed jaw for compression the tissue to be suturing. After verifying the correctness of applyi of the sewing unit, the fuse 16 is removed and movable handle 14 is moved, so it is brought closer to a stationary handle 2. In this case, needle pusher 12 acts on blunt-ends of needles 9 and raises all the needles straight up, the needles sew clamped tissues and get into placed in the top mobile jaw 4 needles receiver 21. Then, turning the screw knob 19 of control of fixation device, stock 17 of needles fixation device is lowered. The needles are fixed in the needles receiver 21. Making sure that the needles are fixed firmly, the screw handle 19 is rotated in the opposite direction, lifting up the entire lifter 21 of having sewed needles with needles until entire pulling needles out of the sutured tissues and enough tension of having sewed threads. Next, the fixing of having sewed threads by one of the methods described above: the tension of spanning loops 30 as the result of rise of the ring 31 by the lift 33 or tension of the rods, to which are attached the tightening loop 30 threads. In the third variant of bonding is necessary to wait 2-3 minutes to complete the polymerization of glue. After that, the rotation of spiral arm 6 in the opposite direction bred jaws 3 and 4 and remove the suturing device. After the removal of the unit the tissue suture is tested. The additional manual bonding of having sewed threads is possible. Then the ends of the threads are cut. Suturing process is complete.

Thus, using the proposed suturing device, any biological tissue or organ other than bone and cartilage can be sutured with soft inert or biologically compatible, including absorbable, threads. Rectilinear motion of direct atraumatic needles provides the least possible trauma of sewed tissues. The presence of absorbable films or plates with locking elements provides additional hemostasis of puncture points and the full seam sealing.

1. Suturing device, comprising a body, two working jaws, one fixed and the second jaw is connected to the body with the ability to move relative to the fixed jaw parallel to the work surface, suturing elements, which are mainly direct atraumatic needle, needles after suture receiver is equipped with a device for needles fixation and a tool for tension and fixing threads, characterized by that in it atraumatic needles are installed in a fixed perpendicular to the immobile jaw working surface, the fimmobile jaw is provided with the pusher moving needle in a rectilinear direction, the receiver of needles after suturing is installed on the mobile jaw.

2. Suturing device according to claim 1, wherein the atraumatic needles are fixed in pairs in two rows on the movable frame in a removable cassette installed in the through-slits made in the fixed jaw and pusher moving needle communicates by its work surface with needle blunt ends and with a movable frame.

3. Suturing device according to claim 2, wherein the needle receiver made in the form of a hollow body and its wall facing the fixed jaw, there are slots to cater for the transmission of the needle, and placed inside the receiver device of fixing the needle after suture-related by stock with a handle managing tool fixation, located at the proximal end of the body.

4. Suturing device according to claim 3, wherein the device of fixing the needle contains a strip of elastic material placed in the hollow body of the needle receiver under the bridge between the slots, and the clamping element is mounted on the end of the stock of fixation device in contact with the strip, providing it with contraction and expansion in the transverse direction at a forward movement of the said stock, and the stopper is provided for fixing the clamping element in the position of elastic strip compression.

5. Suturing device according to claim 3, wherein the device of fixing the needle contains three plates installed in the hollow body of needle receiver perpendicular to the wall with slotted holes for sewing needles, the middle plate located between the rows of sewing needles, and the extreme—on opposite sides of the sewing needles, while all three plates are interconnected by an axle mounted on end walls of the needles receiver, at the ends opposite to the wall with slots, the plates are spring-loaded with each other, and at the end of the fixing device's stock is installed U-shaped clamping element capable of pressing the extreme plates to the middle during the progressive movement of this stock, which is equipped with a stopper foe fixation in the position of the compression of extreme plates to the middle one.

6. Suturing device according to claim 3, wherein the needle fixing device contains two or three plates located in the hollow body of needles receiver, parallel to the wall with slotted holes for sewing needles, with the ability to shift one of the plates relative to the others, and the stock of the fixation device there is an eccentric, which at the turn of this stock around its longitudinal axis moves the specified plate.

7. Suturing device according to claim 3, wherein the needles fixing device contains a plate with support holes for bending the ends of sharp needles, located above the slot in needle receiver, connected by stock of fixation device to the control facility of fixation device, providing compression of bent ends of needles in forward movement of the stock of fixation device, while the stopper is provided for fixing a plate with the support holes in the wall of the needles receiver facing the clamping surface of the movable jaw.

8. Suturing device according to claim 1, wherein the device for tensioning and fixing threads after sewing is done in the form of located on the surface of the movable clamping jaws bilayer film, available for piercing needles, between its layers are placed tightening loops, distributed, taking into account pair-wise coverage of all threads passing through the film when suturing, ends of each loop are directed in opposite sites and pulling these ends leads to the fixation of having sewed threads by tightening loops.

9. Suturing device according to claim 1, characterized in that the device for tensioning and fixing threads after sewing is made in the form of a plate mounted on the clamping surface of the movable jaw, and tubes, done of material available for piercing needles when suturing, filled with biocompatible glue, located along the lines of the passage of two rows of needles during suturing.
